(19) United States
(12) Patent Application Publication
(10) Pub. No.: US 2016/0174584 A1
(43) Pub. Date: Jun. 23, 2016

(54) OXYGEN SCAVENGING STABILIZES COLOR IN MEATS IN LOW OXYGEN

(71) Applicant: Wisconsin Alumni Research Foundation, Madison, WI (US)

(72) Inventors: Mark RICHARDS, Madison, WI (US); Nantawat TATIYABORWORNTHAM, Madison, WI (US); Kathrine H. BAK, Copenhagen (DK)

(73) Assignee: Wisconsin Alumni Research Foundation, Madison, WI (US)

(21) Appl. No.: 14/962,682

(22) Filed: Dec. 8, 2015

Related U.S. Application Data
(60) Provisional application No. 62/104,484, filed on Jan. 16, 2015.
(60) Provisional application No. 62/100,640, filed on Jan. 7, 2015.
(60) Provisional application No. 62/088,954, filed on Dec. 8, 2014.

(51) Int. Cl. A23B 4/22; C12N 9/08; C12N 9/04; B65D 81/26; B65D 81/28

(52) U.S. Cl. CPC A23B 4/22; B65D 81/268; B65D 81/28; C12N 9/0006; C12N 9/0065; A23V 2002/00; C12Y 101/03004; C12Y 111/01006; C12N 2500/02

(57) ABSTRACT

The disclosure provides for compositions and methods for the preservation of meat tissues, including fish, beef, poultry and pork. In particular, the compositions and methods reduce light-induced discoloration.

This application claims benefit of priority to U.S. Provisional Application Ser. Nos. 62/088,954, filed Dec. 8, 2014, 62/100,640, filed Jan. 7, 2015, and 62/104,484, filed Jan. 16, 2015, the entire contents of each application being hereby incorporated by reference.

### BACKGROUND

1. Field

This disclosure relates to composition and methods for the stabilizing color of meat products in low oxygen environments. In particular, the use of oxygen scavenging enzyme solutions is described.

2. Related Art

Food preservation is a complicated process that requires both a means of preventing microbial contamination and a means of preventing the development of off-colors or off-flavors rendering the food unpalatable from visual or flavor aspects. Indeed, off-odor and off-flavor development during refrigerated and frozen storage of fish products is a major obstacle to consumer acceptance. The USDA estimates that more than 96 billion pounds of food in the U.S. were lost by retailers, foodservice, and consumers in 1995, and meat, poultry and fish made up 8.5% of that number—over 8 billion pounds.

In particular, color stability during retail display is a general problem for commercial sliced roast beef products. Currently, some companies manage this problem by packaging sliced roast beef products in a black plastic tray with a black lid, unlike the clear packaging used for other sliced deli products. The goal is to improve the color stability of sliced roast beef so that in the future, it can be packaged in clear packaging without fading significantly during retail display.

### SUMMARY

Thus, in accordance with the present disclosure, there is provided a method of preventing discoloration of a meat product comprising placing a cured meat product in container having a low oxygen environment, wherein said low oxygen environment further comprises a buffered solution comprising at least one oxygen-scavenging enzyme and at least 1% by weight of glucose, and wherein said buffered solution does not come into physical contact with said cured meat product. The at least one oxygen-scavenging enzyme may be glucose oxidase, or both glucose oxidase and catalase, where the weight ratio of glucose oxidase to catalase may be about 10:1. The amount of catalase present may be about 0.1-0.5 mg/ml, and the amount of glucose oxidase present may be about 1.0-5.0 mg/ml, including about 0.1, 0.2, 0.3, 0.4 and 0.5 mg/ml of catalase, and about 1.0, 2.0, 3.0, 4.0 and 5.0 mg/ml of glucose oxidase. The low oxygen environment may further contain nitric oxide, such as wherein the nitric oxide is stably maintained in deoxygenated water. The water containing the nitric oxide may be segregated from the oxygen-scavenging enzyme(s), such as in an air-permeable sachet or packet.

The low oxygen environment may be a nitrogen gas-flushed environment. The container may be formed by a rigid or flexible polymer packaging material. The buffered solution may be located in an air-permeable sachet located in said container, or boated in the rigid or flexible polymer packaging material or a film applied thereto. The meat product may be a cured meat product or an uncured meat product. The meat product may be beef, pork, poultry or fish. The glucose may be present at about 3% by weight of said solution, including about 1-3%, about 1-6%, about 3-6%, about 1-10% or about 6-10% by weight of said solution. The buffered solution may be a phosphate buffered solution, and/or be at about pH 6.2-6.4.

In another embodiment, there is provided a container comprising a low oxygen environment, wherein said low oxygen environment further comprises a buffered solution comprising at least one oxygen-scavenging enzyme and at least 1% by weight of glucose. The at least one oxygen-scavenging enzyme may be glucose oxidase, or both glucose oxidase and catalase, where the weight ratio of glucose oxidase to catalase may be about 10:1. The amount of catalase present may be about 0.1-0.5 mg/ml, and the amount of glucose oxidase present may be about 1.0-5.0 mg/ml, including about 0.1, 0.2, 0.3, 0.4 and 0.5 mg/ml of catalase, and about 1.0, 2.0, 3.0, 4.0 and 5.0 mg/ml of glucose oxidase. The low oxygen environment may further contain nitric oxide, such as wherein the nitric oxide is stably maintained in deoxygenated water. The water containing the nitric oxide may be segregated from the oxygen-scavenging enzyme(s).

The low oxygen environment may be a nitrogen gas-flushed environment. The container may be formed by a rigid or flexible polymer packaging material. The buffered solution may be located in an air-permeable sachet located in said container, or boated in the rigid or flexible polymer packaging material or a film applied thereto. The glucose may be present at about 3% by weight of said solution, including about 1-3%, about 1-6%, about 3-6%, about 1-10% or about 6-10% by weight of said solution. The buffered solution may be a phosphate buffered solution, and/or be at about pH 6.2-6.4. The container may further comprise a meat product located in said low oxygen environment, wherein said meat product does not come into physical contact with said buffered solution. The meat product may be a cured meat product or an uncured meat product.

In yet another embodiment, there is provided a method of preparing a packaged meat comprising (a) providing a container defined by a rigid or flexible polymer packaging material that comprises a buffered solution comprising at least one oxygen-scavenging enzyme and at least 1% by weight of glucose; (b) reducing the oxygen content inside said container to produce a low oxygen environment; (c) introducing said meat product into said low oxygen environment such that said buffered solution does not come into physical contact with the cured meat product; and (d) sealing said container to substantially prevent movement of oxygen into said low oxygen environment. Step (b) may comprise flushing said container with nitrogen gas. The container may also contain nitric oxide, such as wherein the nitric oxide is stably maintained in deoxygenated water. The water containing the nitric oxide may be segregated from the oxygen-scavenging enzyme(s), such as in an air-permeable sachet or packet.

The buffered solution may be located in an air-permeable sachet located in said container. The buffered solution may be located in the rigid or flexible polymer packaging material or a film applied thereto. The at least one oxygen-scavenging enzyme may be glucose oxidase, or may comprise both glucose oxidase and catalase, such as at about about 10:1. The amount of catalase present may be about 0.1-0.5 mg/ml, and the amount of glucose oxidase present may be about 1.0-5.0 mg/ml, including about 0.1, 0.2, 0.3, 0.4 and 0.5 mg/ml of catalase, and about 1.0, 2.0, 3.0, 4.0 and 5.0 mg/ml of glucose oxidase. The glucose may be present at about 3% by weight of said solution, including about 1-3%, about 1-6%, about 3-6%, about 1-10% or about 6-10% by weight of said solution. The meat product may be a cured meat product or an uncured meat product. The meat product may be beef, pork, poultry or fish. The buffered solution may be a phosphate buffered solution, and/or be at about pH 6.2-6.4. The low oxygen environment may be phosphate-free.

Also provided is a buffered solution comprising glucose oxidase and/or catalase, optionally at about a 10:1 ratio. The amount of catalase present may be about 0.1-0.5 mg/ml, and the amount of glucose oxidase present may be about 1.0-5.0 mg/ml, including about 0.1, 0.2, 0.3, 0.4 and 0.5 mg/ml of catalase, and about 1.0, 2.0, 3.0, 4.0 and 5.0 mg/ml of glucose oxidase. The buffered solution may be a phosphate buffered solution, and or be at about 6.2-6.4. The buffered solution may further comprise glucose, such as at about 1%, about 3%, about 1-3%, about 1-6%, about 3-6%, about 1-10% or about 6-10% by weight of said solution.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE FIGURES

The following drawings form part of the present specification and are included to further demonstrate certain aspects of the present disclosure. The disclosure may be better understood by reference to one or more of these drawings in combination with the detailed description of the disclosure that follows.

FIG. 1—CIE a*-values during light display of roast beef samples stored at UW. Dark stored samples were put under light after the color measurement on day 9.

FIG. 2—CIE a*-values during light display of roast beef samples stored under 150 ft. candles for 14 days.

FIG. 3—E4 versus C10 at day 14 of light display.

FIG. 4—OS2 versus E4 at day 14 of light display.

FIG. 5—OS2 versus C10 at day 14 of light display.

FIG. 6—E versus OS after 14+6 days of light display (last six days with some air entering the package where it had been punctured).

### DESCRIPTION OF ILLUSTRATIVE EMBODIMENTS

Discoloration of processed meat products such as deli meat is caused primarily through oxidative damage of the meat. Light can enhance the discoloration process in the package meat, especially cured roast beef. Because of this, most packaged roast beef slices are sold in darkened packages, making it harder to capture the eye of the consumer. However, the discoloration of the meat is a turnoff to the consumer, and presently darkened packages are the only feasible way to prevent light-induced discoloration of the meat.

The present disclosure describes the use a combination of small molecules and enzymes to prevent oxygen-induced discoloration in packaged roast beef. Meat packaging uses nitrogen flushing during packaging. However, a small amount of O₂ is present in the nitrogen lines and the nitrogen flush cannot displace all the O₂ in contact with the meat during the flushing and packaging process. The inventor hypothesized that glucose, glucose oxidase and catalase could scavenge trace O₂ amounts during meat packaging resulting in better flavor and color. These reagents would be used in the package and not in the food itself.

The inventors created a preservation package containing enzyme stock solution and a glucose solution. The enzyme stock solution contains catalase and glucose oxidase. The preservation package incorporates these components in an air-permeable bag, which was introduced into the meat package. After storage of meat under refrigeration and light after 14 days, the container incorporating the preservation package showed dramatically less discoloration than the control package. Thus, the data presented below indicate that this method is a viable approach to the preservation of meat, in particular the reduction of light-induced discoloration in cured meats.

These and other aspects of the disclosure are described in detail below.

### I. OXYGEN SCAVENGING ENZYMES

Oxygen scavengers are well known in the food industry. These include both enzymatic and non-enzymatic agents. Regardless, oxygen scavenger must satisfy several requirements:
be harmless to the human body,
absorb oxygen at an appropriate rate,
not produce toxic substances or unfavorable gas or odor,
be compact in size,
show a constant quality and performance,
absorb a large amount of oxygen,
be economically priced,
not be inadvertently ingested by the consumer if the person does not see the scavenger packet for example, and
not discolor the meat product if the scavenger packet comes in contact with the meat for example
   See Nakamura and Hoshino (1983); Abe (1994); Rooney (1995). Enzymatic oxygen scavengers include glucose oxidase, catalase and ethanol oxidase.

Englander et al. (1987) found that a glucose oxidase/catalase enzyme system can deplete residual oxygen from experimental solutions via the following reactions:

2O₂+2β-D-glucose→2gluconolactone+2H₂O₂ (glucose oxidase catalyzes the reaction) 2gluconolactone+2H₂O→2gluconic acid (spontaneous hydrolysis)

2H₂O₂→2H₂O+O₂ (catalase catalyzes the reaction)

The net reaction therefore is:

O₂+2β-D-glucose→2gluconic acid

However, this enzyme system has not been tested for depleting residual oxygen from meat packaging. Such oxygen depletion, if possible, could delay color fading significantly, since the combination of oxygen and light is known to cause rapid discoloration of cured meat products (Andersen, Bertelsen, Boegh-Soerensen, Shek, & Skibsted, 1988; Andersen & Rasmussen, 1992). The reaction may be enhanced by the inclusion of NO, but the mechanism by which NO may accelerate O₂ depletion is not clear. However, the enzyme system shows little O₂ depletion in the absence of light (sample E DC) compared to in the presence of light (sample E, see Table 1). Light will cause NO to dissociate from heme pigments in cured meat so it is plausible that NO enhances O₂ depletion in the presence of the enzyme system at low O₂ partial pressure. The competiting reaction due to light that will deplete O₂ is light-mediated conversion of O₂ to singlet O₂ that then reacts with other biomolecules. However, formation of substantial amounts of singlet O₂ should result in elevated lipid oxidation which was not observed (see Table 2).

A. Glucose oxidase

The glucose oxidase enzyme (GOx) is an oxido-reductase that catalyses the oxidation of glucose to hydrogen peroxide and D-glucono-δ-lactone. In cells, it aids in breaking the sugar down into its metabolites.

Glucose oxidase is widely used for the determination of free glucose in body fluids (diagnostics), in vegetal raw material, and in the food industry. It also has many applications in biotechnologies, typically enzyme assays for biochemistry including biosensors in nanotechnologies. It is often extracted from Aspergillus niger.

GOx is a dimeric protein, the 3D structure of which has been elucidated. The active site where glucose binds is in a deep pocket. The enzyme, like many proteins that act outside of cells, is covered with carbohydrate chains.

At pH 7, glucose exists in solution in cyclic hemiacetal form as 63.6% β-D-glucopyranose and 36.4% α-D-glucopyranose, the proportion of linear and furanose form being negligible. The glucose oxidase binds specifically to β-D-glucopyranose and does not act on α-D-glucose. It is able to oxidise all of the glucose in solution because the equilibrium between the α and β anomers is driven towards the β side as it is consumed in the reaction.

Glucose oxidase catalyzes the oxidation of β-D-glucose into D-glucono-1,5-lactone, which then hydrolyzes to gluconic acid.

In order to work as a catalyst, GOx requires a cofactor, flavin adenine dinucleotide (FAD). FAD is a common component in biological oxidation-reduction (redox reactions). Redox reactions involve a gain or loss of electrons from a molecule. In the GOx-catalyzed redox reaction, FAD works as the initial electron acceptor and is reduced to FADH₂. Then FADH₂ is oxidized by the final electron acceptor, molecular oxygen (O₂), which can do so because it has a higher reduction potential. O₂ is then reduced to hydrogen peroxide (H₂O₂). The reaction is as follows:

FAD+glucose→FADH2+gluconolactone

FADH2+O2→FAD+H2O2 (this reaction requires glucose oxidase)

### See Prabhakar et al. (2003).

Glucose oxidase is widely used coupled to peroxidase reaction that visualizes colorimetrically the formed H₂O₂, for the determination of free glucose in sera or blood plasma for diagnostics, using spectrometric assays manually or with automated procedures, and even point of use rapid assays. Similar assays allow one to monitor glucose levels in fermentation, bioreactors, and to control glucose in vegetal raw material and food products. In manufacturing, GOx is used as an additive thanks to its oxidizing effects: it prompts for stronger dough in bakery, replacing oxidants such as bromate. It also helps remove oxygen from food packaging, or D-glucose from egg white to prevent browning. Glucose oxidase is found in honey and acts as a natural preservative. GOx at the surface of the honey reduces atmospheric O₂ to hydrogen peroxide (H₂O₂), which acts as an antimicrobial barrier. GOx similarly acts as a bactericide in many cells (fungi, immune cells).

Notatin, extracted from antibacterial cultures of Penicillium notatum, was originally named Penicillin A, but was renamed to avoid confusion with penicillin. Notatin was shown to be identical to Penicillin B and glucose oxidase, enzymes extracted from other molds besides P. notatum; it is now generally known as glucose oxidase.

Early experiments showed that notatin exhibits in vitro antibacterial activity (in the presence of glucose) due to hydrogen peroxide formation. In vivo tests showed that notatin was not effective in protecting rodents from Streptococcus haemolyticus, Staphylococcus aureus, or salmonella, and caused severe tissue damage at some doses.

The enzyme activity for glucose oxidase (Aspergillus niger Type VII) used in the experiments was 266,660 units/g solid for the studies in Tables 1-3, and 149,800 U/g solid for Table 4.

B. Catalase

Catalase is a common enzyme found in nearly all living organisms exposed to oxygen (such as vegetables, fruit or animals). It catalyzes the decomposition of hydrogen peroxide to water and oxygen. It is a very important enzyme in protecting the cell from oxidative damage by reactive oxygen species (ROS) Likewise, catalase has one of the highest turnover numbers of all enzymes; one catalase molecule can convert millions of molecules of hydrogen peroxide to water and oxygen each second.

Catalase is a tetramer of four polypeptide chains, each over 500 amino acids long. It contains four porphyrin heme (iron) groups that allow the enzyme to react with the hydrogen peroxide. The optimum pH for human catalase is approximately 7, and has a fairly broad maximum (the rate of reaction does not change appreciably at pHs between 6.8 and 7.5). The pH optimum for other catalases varies between 4 and 11 depending on the species. The optimum temperature also varies by species.

The reaction of catalase in the decomposition of hydrogen peroxide in living tissue:

2H₂O₂→2H₂O+O₂

The presence of catalase in a microbial or tissue sample can be tested by adding a volume of hydrogen peroxide and observing the reaction. The formation of bubbles, oxygen, indicates a positive result. This easy assay, which can be seen with the naked eye, without the aid of instruments, is possible because catalase has a very high specific activity, which produces a detectable response. Catalase can also catalyze the oxidation, by hydrogen peroxide, of various metabolites and toxins, including formaldehyde, formic acid, phenols, acetaldehyde and alcohols. It does so according to the following reaction:

H₂O₂+H₂R→2H₂O+R

The exact mechanism of this reaction is not known.

Any heavy metal ion (such as copper cations in copper(II) sulfate) can act as a noncompetitive inhibitor of catalase. Furthermore, the poison cyanide is a competitive inhibitor of catalase at high concentrations of hydrogen peroxide. Three-dimensional protein structures of the peroxidated catalase intermediates are available at the Protein Data Bank. This enzyme is commonly used in laboratories as a tool for learning the effect of enzymes upon reaction rates.

Hydrogen peroxide is a harmful byproduct of many normal metabolic processes; to prevent damage to cells and tissues, it must be quickly converted into other, less dangerous substances. To this end, catalase is frequently used by cells to rapidly catalyze the decomposition of hydrogen peroxide into less-reactive gaseous oxygen and water molecules.

The true biological significance of catalase is not always straightforward to assess: Mice genetically engineered to lack catalase are phenotypically normal, indicating this enzyme is dispensable in animals under some conditions. A catalase deficiency may increase the likelihood of developing type 2 diabetes. Some humans have very low levels of catalase (acatalasia), yet show few ill effects. The predominant scavengers of H₂O₂ in normal mammalian cells are likely peroxiredoxins rather than catalase.

Human catalase works at an optimum temperature of 45° C. In contrast, catalase isolated from the hyperthermophile archaeon Pyrobaculum calidifontis has a temperature optimum of 90° C. Catalase is usually located in a cellular, bipolar environment organelle called the peroxisome. Peroxisomes in plant cells are involved in photorespiration (the use of oxygen and production of carbon dioxide) and symbiotic nitrogen fixation (the breaking apart of diatomic nitrogen (N₂) to reactive nitrogen atoms). Hydrogen peroxide is used as a potent antimicrobial agent when cells are infected with a pathogen. Catalase-positive pathogens, such as Mycobacterium tuberculosis, Legionella pneumophila, and Campylobacter jejuni, make catalase to deactivate the peroxide radicals, thus allowing them to survive unharmed within the host.

Catalase contributes to ethanol metabolism in the body after ingestion of alcohol, but it only breaks down a small fraction of the alcohol in the body. Catalase is used in the food industry for removing hydrogen peroxide from milk prior to cheese production. Another use is in food wrappers where it prevents food from oxidizing. Catalase is also used in the textile industry, removing hydrogen peroxide from fabrics to make sure the material is peroxide-free.

The catalase (bovine liver) used in the experiments described in the Examples was 2350 Units/mg solid.

A minor use is in contact lens hygiene. A few lens-cleaning products disinfect the lens using a hydrogen peroxide solution; a solution containing catalase is then used to decompose the hydrogen peroxide before the lens is used again. Recently, catalase has also begun to be used in the aesthetics industry. Several mask treatments combine the enzyme with hydrogen peroxide on the face with the intent of increasing cellular oxygenation in the upper layers of the epidermis.

The catalase test is also one of the main three tests used by microbiologists to identify species of bacteria. The presence of catalase enzyme in the test isolate is detected using hydrogen peroxide. If the bacteria possess catalase (i.e., are catalase-positive), when a small amount of bacterial isolate is added to hydrogen peroxide, bubbles of oxygen are observed.

### II. MEAT PROCESSING

Meat is produced by killing an animal and cutting flesh out of it. These procedures are called slaughter and butchery, respectively. The general process for preparing meat for consumption involves the steps of transport, slaughter, dressing & cutting, conditioning, treatment with additives, preservation and packaging. These steps are described below.

A. Transport

Upon reaching a predetermined age or weight, livestock are usually transported en masse to the slaughterhouse. Depending on its length and circumstances, this may exert stress and injuries on the animals, and some may die en route. Unnecessary stress in transport may adversely affect the quality of the meat. In particular, the muscles of stressed animals are low in water and glycogen, and their pH fails to attain acidic values, all of which results in poor meat quality. Consequently, and also due to campaigning by animal welfare groups, laws and industry practices in several countries tend to become more restrictive with respect to the duration and other circumstances of livestock transports.

B. Slaughter

Animals are usually slaughtered by being first stunned and then exsanguinated (bled out). Death results from the one or the other procedure, depending on the methods employed. Stunning can be effected through asphyxiating the animals with carbon dioxide, shooting them with a gun or a captive bolt pistol, or shocking them with electric current. In most forms of ritual slaughter, stunning is not allowed.

Draining as much blood as possible from the carcase is necessary because blood causes the meat to have an unappealing appearance and is a very good breeding ground for microorganisms. The exsanguination is accomplished by severing the carotid artery and the jugular vein in cattle and sheep, and the anterior vena cava in pigs.

C. Dressing & Cutting

After exsanguination, the carcase is dressed; that is, the head, feet, hide (except hogs and some veal), excess fat, viscera and offal are removed, leaving only bones and edible muscle. Cattle and pig carcases, but not those of sheep, are then split in half along the mid ventral axis, and the carcase is cut into wholesale pieces. The dressing and cutting sequence, long a province of manual labor, is progressively being fully automated.

D. Conditioning

Under hygienic conditions and without other treatment, meat can be stored at above its freezing point (−1.5° C.) for about six weeks without spoilage, during which time it undergoes an aging process that increases its tenderness and flavor.

During the first day after death, glycolysis continues until the accumulation of lactic acid causes the pH to reach about 5.5. The remaining glycogen, about 18 g per kg, is believed to increase the water-holding capacity and tenderness of the flesh when cooked. Rigor mortis sets in a few hours after death as ATP is used up, causing actin and myosin to combine into rigid actomyosin and lowering the meat's water-holding capacity, causing it to lose water (“weep”). In muscles that enter rigor in a contracted position, actin and myosin filaments overlap and cross-bond, resulting in meat that is tough on cooking—hence again the need to prevent pre-slaughter stress in the animal.

Over time, the muscle proteins denature in varying degree, with the exception of the collagen and elastin of connective tissue, and rigor mortis resolves. Because of these changes, the meat is tender and pliable when cooked just after death or after the resolution of rigor, but tough when cooked during rigor. As the muscle pigment myoglobin denatures, its iron oxidates, which may cause a brown discoloration near the surface of the meat. Ongoing proteolysis also contributes to conditioning. Hypoxanthine, a breakdown product of ATP, contributes to the meat's flavor and odor, as do other products of the discomposition of muscle fat and protein.

E. Treatment with Additives

When meat is industrially processed in preparation of consumption, it may be enriched with additives to protect or modify its flavor or color, to improve its tenderness, juiciness or cohesiveness, or to aid with its preservation. Meat additives include the following:
Salt is the most frequently used additive in meat processing. It imparts flavor but also inhibits microbial growth, extends the product's shelf life and helps emulsifying finely processed products, such as sausages. Ready-to-eat meat products normally contain about 1.5 to 2.5 percent salt.
Nitrite is used in curing meat to stabilize the meat's color and flavor, and inhibits the growth of spore-forming microorganisms such as C. botulinum. The use of nitrite's precursor nitrate is now limited to a few products such as dry sausage, prosciutto or parma ham.
Phosphates used in meat processing are normally alkaline polyphosphates such as sodium tripolyphosphate. They are used to increase the water-binding and emulsifying ability of meat proteins, but also limit lipid oxidation and flavor loss, and reduce microbial growth.
Erythorbate or its equivalent ascorbic acid (vitamin C) is used to stabilize the color of cured meat.
Sweeteners such as sugar or corn syrup impart a sweet flavor, bind water and assist surface browning during cooking in the Maillard reaction.
Seasonings impart or modify flavor. They include spices or oleoresins extracted from them, herbs, vegetables and essential oils.
Flavorings such as monosodium glutamate impart or strengthen a particular flavor.
Tenderizers break down collagens to make the meat more palatable for consumption. They include proteolytic enzymes, acids, salt and phosphate.
Dedicated antimicrobials include lactic, citric and acetic acid, sodium diacetate, acidified sodium chloride or calcium sulfate, cetylpyridinium chloride, activated lactoferrin, sodium or potassium lactate, or bacteriocins such as nisin.
Antioxidants include a wide range of chemicals that limit lipid oxidation, which creates an undesirable “off flavor,” in precooked meat products.
Acidifiers, most often lactic or citric acid, can impart a tangy or tart flavor note, extend shelf-life, tenderize fresh meat or help with protein denaturation and moisture release in dried meat. They substitute for the process of natural fermentation that acidifies some meat products such as hard salami or prosciutto.

F. Curing

Curing is any of various food preservation and flavoring processes of foods such as meat, fish and vegetables, by the addition of a combination of salt, nitrates, nitrite or sugar. Many curing processes also involve smoking, the process of flavoring, or cooking. The use of food dehydration was the earliest form of food curing.

Sodium nitrite is the primary ingredient used in meat curing. Sodium nitrite can be added to the meat formulation as a pure chemical at low levels (e.g., 156 ppm) or sodium nitrate in vegetables (e.g., celery powder) can be converted to sodium nitrite by a reducing enzyme in a starter culture (e.g., Micrococcus). Curing is often done in the presence of salt (sodium chloride). Added salt at pH values above 5.3 increases water binding. At pH values near 5.0, salt aids in water removal from the the meat (e.g., hard salami). In addition, salt causes the soluble meat proteins to come to the surface of the meat particles within sausages. These proteins coagulate when the sausage is heated, helping to hold the sausage together. Salt increases the oxidation process in the absence of sodium nitrite. Curing with sodium nitrite however effectively inhibits the meat from going rancid during storage. The sugar added to meat for the purpose of sweetness comes in many forms, including honey, corn syrup solids, and maple syrup. Sugar complements the flavor of the salt. Sugar also contributes to the growth of beneficial bacteria in fermented and naturally cured meat products like Lactobacillus and Micrococcus, by feeding them.

Nitrates and nitrites not only help kill bacteria, but also produce a characteristic flavor and give meat a pink or red color. Nitrate (NO₃⁻), generally supplied by sodium nitrate or potassium nitrate, is used as a source for nitrite (NO₂⁻). The nitrite further breaks down in the meat into nitric oxide (NO), which then binds to the iron atom in the center of myoglobin's heme group, reducing oxidation and causing a reddish-brown color (nitrosomyoglobin) when raw, and the characteristic cooked-ham pink color (nitrosohemochrome or nitrosyl-heme) when cooked. The addition of ascorbate or erythorbate to cured meat reduces formation of nitrosamines, but increases the nitrosylation of iron.

The use of nitrates in food preservation is controversial. This is due to the potential for the formation of nitrosamines when nitrates are present in high concentrations and the product is cooked at high temperatures. The effect is seen for red or processed meat, but not for white meat or fish. The production of carcinogenic nitrosamines can be potently inhibited by the use of the antioxidants Vitamin C and the alpha-tocopherol form of Vitamin E during curing. Under simulated gastric conditions, nitrosothiols rather than nitrosamines are the main nitroso species being formed. The usage of either compound is therefore regulated; for example, in the United States, the concentration of nitrites and nitrates is generally limited to 200 ppm or lower. Added nitrates are no longer allowed in bacon, and erythorbate or ascorbate are required as curing accelerators in bacon to decrease risk of nitrosamine formation. Further, specific cooking instructions for bacon (340° F. for 3 minutes per side) are provided by the USDA Code of Federal Regulations (CFR 9 242.22) with an upper limit of added nitrite at 120 ppm. Nitrite is considered irreplaceable in the prevention of botulinum poisoning from consumption of cured dry sausages by preventing spore germination.

Meat can also be preserved by “smoking,” which means exposing it to smoke from burning or smoldering plant materials, usually wood. If the smoke is hot enough to slow-cook the meat, it will also keep it tender. One method of smoking calls for a smokehouse with damp wood chips or sawdust. In North America, hardwoods such as hickory, mesquite and maple are commonly used for smoking, as are the wood from fruit trees such as apple, cherry, and plum, and even corncobs.

Smoking helps seal the outer layer of the food being cured, making it more difficult for bacteria to enter. It can be done in combination with other curing methods such as salting. Common smoking styles include hot smoking, smoke roasting (pit barbecuing) and cold smoking. Smoke roasting and hot smoking cook the meat while cold smoking does not. If the meat is cold smoked, it should be dried quickly to limit bacterial growth during the critical period where the meat is not yet dry. This can be achieved, as with jerky, by slicing the meat thinly.

G. Preservation and Packaging

There are two types of deterioration in meats, microbial spoilage and chemical oxidation. The spoilage of meat occurs, if untreated, in a matter of hours or days and results in the meat becoming unappetizing, poisonous or infectious. Spoilage is caused by the practically unavoidable infection and subsequent decomposition of meat by bacteria and fungi, which are borne by the animal itself, by the people handling the meat, and by their implements. Meat can be kept edible for a much longer time—though not indefinitely—if proper hygiene is observed during production and processing, and if appropriate food safety, food preservation and food storage procedures are applied. Chemical oxidation is a process by which oxygen adds to proteins and fats in meat which results in a complex array of further reactions that compromise texture, flavor, odor, and appearance of the meat product. Chemical oxidation is inhibited during storage by natural and synthetic antioxidants such as plant extracts or propyl gallate. Curing is also effective at inhibiting chemical oxidation in meat during storage. Without the application of preservatives and stabilizers, the fats in meat may also begin to rapidly decompose after cooking or processing, leading to an objectionable taste known as warmed over flavor.

### III. PRESERVATION COMPOSITIONS

In aspects of the disclosure, there are provided preservation compositions, e.g., solutions, containing various agents useful in preventing the discoloration of meats. The components of the compositions may include oxygen scavenging enzymes and buffered solutions, and optionally glucose. The oxygen scavenging enzymes may be glucose oxidase and catalase, and may be present at a ratio of 20:1, 10:1 or 5:1 for glucose oxidase:catalase. The solution may contain, in particular, about 1.0 mg/ml glucose oxidase, and about 0.1 mg/ml catalase. The glucose may be present at about 1-5% by weight of said solution, or about 3%. The buffered solution may be a phosphate buffered solution. The pH of the buffered solution may be about 6-6.6, or about 6.2-6.4, or about 6.3.

### IV. METHODS OF PRESERVING MEATS

The methods of the present disclosure may be applied to a wide variety of cured and uncured meat products. In general, the methods simply require a meat product to be placed in a sealed environment with the preservation compositions described above. The compositions should not come into direct contact with the meat products, but must be in connection with the gaseous environment within a sealed container. Rather, they a generally position to be retained in a gas permeable device, such as a sachet or bag, that is on the interior of a package containing the meat. Alternatively, the compositions may be impregnated into the package material itself.

As part of a production process, meats will be provided in appropriate form for final packaging. This includes any pretreatment (curing, smoking, etc.) and processing (removal of fat, cutting, slicing, etc.). The packaging materials containing the preservation solutions will then be provided and meat products inserted. Finally, the packaging will be seal using one or more mechanical, chemical or thermal processes. Prior to final sealing, the gaseous content of the packaging may be altered to reduce the gas and/or oxygen content, such as by subjecting the package to vacuum, or by flushing with another gas, such as nitrogen. Finally, the sealed product will be subject to labeling and final commercial packaging (e.g., boxing).

### V. MEAT PRODUCTS FOR PRESERVATION

A. Meat Tissues

The present disclosure may be applied to virtually any meat product. Examples include avian tissue, amphibian tissue (frog), fish tissue, shellfish tissue, and red meat. Red meat includes pork tissue, beef tissue, bison tissue, mutton tissue, elk tissue, deer tissue, rabbit tissue. Avian tissue includes quail, chicken, dove, turkey, or ostrich. Shellfish tissue includes lobster, shrimp, crab, prawn, crawfish and molluscs (squid, octopus). Fish tissue includes capelin, cod, flounder, grouper, halibut, swordfish, mahi mahi, salmon, redfish, sole, whitefish, tuna, amberjack, char, sea bass, striped bass, sunfish, crappie, catfish, bream, turbot, snapper, carp, chub, drum, haddock, hake, herring, mackerel, monkfish, mullet, rockfish, pollock, pompano, pufferfish, sardine, scrod, skate, sturgeon, tilapia, welk, and whiting. Another fish product is fish eggs, such as caviar.

B. Pet Food

Pet food is plant or animal material intended for consumption by pets. Typically sold in pet stores and supermarkets, it is usually specific to the type of animal, such as dog food or cat food. Most meat used for nonhuman animals is a byproduct of the human food industry, and is not regarded as “human grade.” Four companies—Procter & Gamble, Nestlé, Mars, and Colgate-Palmolive—are thought to control 80% of the world's pet-food market, which in 2007 amounted to US$45.12 billion for cats and dogs alone.

Some types of pet foods—particularly those for dogs and cats—use meat products. Indeed, cats are obligate carnivores, though most commercial cat food contains both animal and plant material supplemented with vitamins, minerals and other nutrients. While recommendations differ on what diet is best for dogs, some form of meat product is included in the food bet that dry form, also known as kibble, or wet, canned form. Also, raw feeding is the practice of feeding domestic dogs and cats a diet consisting primarily of uncooked meat and bones. Supporters of raw feeding believe the natural diet of an animal in the wild is its most ideal diet and try to mimic a similar diet for their domestic companions.

C. Rendered Products

Edible rendering processes are basically meat processing operations and produce lard or edible tallow for use in food products. Edible rendering is generally carried out in a continuous process at low temperature (less than the boiling point of water). The process usually consists of finely chopping the edible fat materials (generally fat trimmings from meat cuts), heating them with or without added steam, and then carrying out two or more stages of centrifugal separation. The first stage separates the liquid water and fat mixture from the solids. The second stage further separates the fat from the water. The solids may be used in food products, pet foods, etc., depending on the original materials. The separated fat may be used in food products, or if in surplus, it may be diverted to soap making operations. Most edible rendering is done by meat packing or processing companies.

One edible product is greaves, which is the unmeltable residue left after animal fat has been rendered. An alternative process cooks slaughterhouse offal to produce a thick, lumpy “stew” which is then sold to the pet food industry to be used principally as tinned cat and dog foods.

Materials that for aesthetic or sanitary reasons are not suitable for human food are the feedstocks for inedible rendering processes. Much of the inedible raw material is rendered using the “dry” method. This may be a batch or a continuous process in which the material is heated in a steam-jacketed vessel to drive off the moisture and simultaneously release the fat from the fat cells. The material is first ground, next heated to release the fat and drive off the moisture, percolated to drain off the free fat, and then more fat is pressed out of the solids, which at this stage are called “cracklings” or “dry-rendered tankage.” The cracklings are further ground to make meat and bone meal. A variation on a dry process involves finely chopping the material, fluidizing it with hot fat, and then evaporating the mixture in one or more evaporator stages. Some inedible rendering is done using a wet process, which is generally a continuous process similar in some ways to that used for edible materials. The material is heated with added steam and then pressed to remove a water-fat mixture which is then separated into fat, water and fine solids by stages of centrifuging and/or evaporation. The solids from the press are dried and then ground into meat and bone meal. Most independent renderers process only inedible material.

Any of the aforementioned rendered products may be treated in accordance with the present disclosure to improve stability.

D. Cured Meats

A wide variety of meats are subject to curing processes. These include fish and seafood selected from African longfin eel, Arbroath smokie, Atlantic mackerel, Bokkoms, Bonga shad, Buckling (fish), Cakalang fufu, Caviar substitutes, Lysekil Caviar, Cod (food), Finnan haddie, Goldeye, Gwamegi, Traditional Grimsby smoked fish, Trout, Herring, Bloater (herring), Blueback herring, Kipper, Craster kipper, Katsuobushi, Saramur\check{a}, Sardine, Smoked salmon, Lox, Smörgåskaviar, Smoked oyster, and Smoked scallop.

Cured meat products include bacon, roast beef, roast pork, chicken, smoke quail, Brési, brisket, Cecina, various forms of Charcuterie, Chaudin, Dutch Loaf, Elenski but, Flurgönder, Gammon, Grjúpán, Hangikjöt, Horse meat, Qarta, Zhal, Jeju Black Pig, Jerky, Kassler, Pastrami, Pickled pigs feet, Pig candy, Pitina, Pork jowl, Oreilles de crisse, Pork tail, Salo, Schäufele, Se'i, Smalahove, Speck, Speck Alto Adige PGI, Tyrolean Speck, Suho meso, Szalonna, turkey, duck, ham, Ammerländer Schinken, Ham hock, and Eisbein.

A variety of sausages include Ahle Wurst, Amsterdam ossenworst, Andouille, Bierwurst, Bockwurst, Bologna sausage, Breakfast sausage, Cabanossi, Chorizo, Ciauscolo, Debrecener, Embutido, Farinheira, Isterband, Kielbasa, Knackwurst, Knipp, Kochwurst, Kohlwurst, Krakowska, Kulen, Lebanon bologna, Linguiça, Liverwurst, Braunschweiger, Loukaniko, Lucanica, Mettwurst, Morteau sausage, N\check{a}dlac sausage, Pinkel, Rookworst, Salami, Skilandis, Sremska kobasica, Summer sausage, Teewurst, Vienna sausage, and Winter salami.

### VI. PACKAGING MATERIALS

In one aspect, the disclosure provides for packaging materials for storing cured meats. The packaging materials are typically made of transparent or semi-transparent, rigid, semi-rigid or flexible plastic materials common in the food packaging industry. Polypropylene and PTE are typical polymers used in plastic packaging. Post-consumer recycled plastics are also becoming more commonly utilized. The containers may be subject to heat sealing or pressure sealing.

In accordance with the present disclosure, the container with include an air-permeable chamber within the interior of the container. One form of such a chamber is a sachet or plastic bag. The bag/sachet will typically contains a buffered solution with one or more scavenging enzymes. The sachet/bag may be attached to an interior surface of the container with an adhesive avoid contact with a meat product. In an alternative embodiment, the buffered solution may be applied to the interior suface of the container, while maintaining any meat product separate from the solution. The interior environment of the container may be low oxygen environment, such as one that has been nitrogen gas-flushed. And, obviously, the container once in use will contain a meat product.

### VII. EXAMPLES

The following examples are included to demonstrate particular embodiments of the disclosure. It should be appreciated by those of skill in the art that the techniques disclosed in the examples which follow represent techniques discovered by the inventor to function well in the practice of the disclosure, and thus can be considered to constitute particular modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Materials and Methods

Solutions. All solutions were prepared fresh for each trial. A 0.05 M sodium phosphate buffer of pH 6.3 at 4° C. and ionic strength 0.067 M was prepared by dissolving 0.0082 mol of acid component and 0.0017 mol of basic component in 200 ml pure water and titrating to pH 6.25 at 20° C. with monovalent strong acid or base as needed. Using the sodium phosphate buffer, solutions of 0.30% glucose, 0.33% glucose, and 3.0% glucose, as well as an enzyme stock solution were prepared. The enzyme stock solution had a final concentration of 10 mg/mL of glucose oxidase and 1 mg/mL of catalase in all trials.

Preliminary study. In the preliminary study, the effect of the enzyme combination glucose oxidase and catalase in a glucose solution made with phosphate buffer was tested. This was done by adding enzyme and glucose solution to Mason jars with or without subsequent flushing with N2 and compare to the effect of flushing with N2 alone. Flushing was done through a 25G 1.5″ (3.81 cm) needle at a pressure of 30 kPa.

Briefly, Mason jars were subjected to one of three treatments (T1 through T3) with six jars per treatment for a total of 18 jars. Two holes were drilled in each of the lids of the Mason jars, and then covered with a sticky nickel each. The three treatments were as follows:
T1: To the Mason jars was added 900 μL 0.33% glucose and 100 μL enzyme stock solution.
T2: To the Mason jars was added 900 μL 0.33% glucose and 100 μL enzyme stock solution. The jars were then flushed with N2 for 5 min through the holes drilled in the lids (sticky nickels were applied immediately after the flushing).
T3: To the Mason jars was added 1000 μL 0.30% glucose. The jars were then flushed with N2 for 5 min through the holes drilled in the lids (sticky nickels were applied immediately after the flushing).
   All jars were stored in a cold room (4° C.) with light display through day 10 of storage and then on a bench in the lab (20° C.) until day 21 of storage. Residual O₂ was measured on days 1, 5, 10, and 21 of storage.

First roast beef trial. In the first roast beef trial, the glucose/catalase enzyme system was tested at two different levels on roast beef sliced and packaged in samples of 7 oz. in transparent plastic trays. Enzyme solution was added to the roast beef samples by filling very small sachets made of oxygen permeable material with 1 mL of solution and carefully placing the sachet on top of the meat just before sealing the lid of the package.

The treatments tested were:
T1: Control (no sachet)
T2: High enzyme/low glucose: 100 μL enzyme stock solution+900 μL 0.3% glucose solution
T3: Low enzyme/high glucose: 10 μL enzyme stock solution+990 μL 3.0% glucose solution
   From each treatment, five samples were subjected to light display at two different facilities. At both facilities, color was measured via a Minolta colorimeter as well as by visual comparison to a sixth sample of the corresponding treatment stored in the dark. At one facility, the light intensity was approx. 150 ft. candles, and the temperature approx. 1-4° C., and color was measured initially as well as on days 3, 6, and 9 of storage for samples stored under light. The study was subsequently continued by putting the dark stored samples under light at this point and measuring color on days 12 and 15. At the other facility, color was measured initially as well as on days 3, 6, 9, 12, and 15 of storage under light.

Second roast beef trial. After less than promising results of the first roast beef trial, it was decided to repeat the experiment with slightly different treatments. The roast beef samples were sliced and packaged similarly to the first trail. For the second trial, the treatments and abbreviations were as follows:
C: Control (no sachet)
C SS: Control, single slice (no sachet)
E: High enzyme/high glucose, i.e., 100 μL enzyme stock solution+900 μL 3.0% glucose solution (0.1 mg/mL catalase and 1.0 mg/mL glucose oxidase in the sachets)
OS: Oxygen scavenger sticker (positive control)
   A total of 36 roast beef samples were prepared according to the following labelling:
C1-C6 DA: Control samples stored dark always
C7-C11: Control samples stored under light
C12 SS-C17 SS: Single slice control samples stored under light
E1-E10: Enzyme samples stored under light
OS1-OS6: Oxygen scavenger samples stored under light (OS 3, 4, and 5 contained one sticker, OS 1, 2, and 6 contained two stickers)
C18 DC: Control sample stored in the dark but color measured at each time point
E11-E12 DC: Enzyme samples stored in the dark but color measured at each time point
   After packaging, all samples were stored in the dark for five days. Then, samples for light display were stored under light at 150 ft. candles at 1-4° C. for 14 days. Color was measured via a Minolta colorimeter on days 1, 2, 3, 4, 5, 7, 10, and 14 of storage. On day 14, headspace O2 was measured on all samples except for the C DA samples.

Furthermore, three samples each of C SS (brown), C DA (red), and E (intermediate) were sampled for hexanal (1 g per sample) and NOx measurement (5 g per sample). Sampled were C13 SS, C15 SS, C16 SS, C 1 DA, C4 DA, C6 DA, E4, E5, and E9. All samples were randomly chosen, except for E4, which was chosen because it was the best looking enzyme sample at the end of the storage period. Slices from the surface were used for these analyses, and the same slices were sampled for both analyses.

### Example 2

### Results and Discussion

Preliminary study. Overall, the preliminary study in a model system indicated that a glucose oxidase/catalase enzyme system could be somewhat efficient in depleting residual O₂ from a system already low in O₂. T1 (enzyme, no N₂-flushing) had the highest O₂ level, which was close to the atmospheric level. Both T2 (enzyme+N₂ flushing) and T3 (N₂ flushing) had somewhat lower levels, on average 3.0-3.5% throughout the storage period for T2 and 6.0%-9.7% for T3. These levels were shown by T-test to be significantly different on day 10 and day 21 (it was only possible to measure very few samples on day 1 and day 5).

First roast beef trial. Already on day 3 of storage, the color of the samples subjected to light display differed significantly from the dark stored samples, and were borderline unacceptable to consumers, as judged by visual rating.

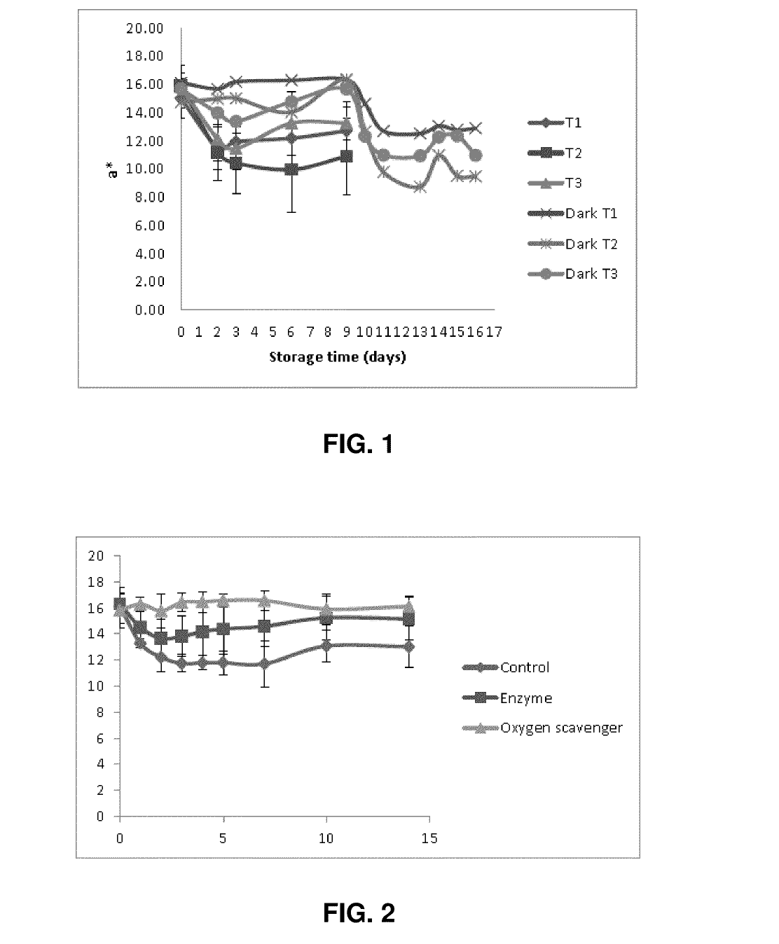

It is seen from FIG. 1 that initial a*-values were fairly similar for all samples. Samples stored under light at one facility experienced a rapid decline in a*, which supported the visual observations. Similar results were found for the light storage conducted at the other facility (results not shown). Towards the end of the light display (FIG. 1), samples with the high enzyme/low glucose level (T2) seemed to perform slightly worse than the control (T1) and the low enzyme/high glucose samples (T3). On a positive note, the sachets appeared to hold the liquid in rather well, as long as they were closed carefully with no liquid in the lining. Most samples had >0.9 mL of liquid left in the sachets at the end of the storage period.

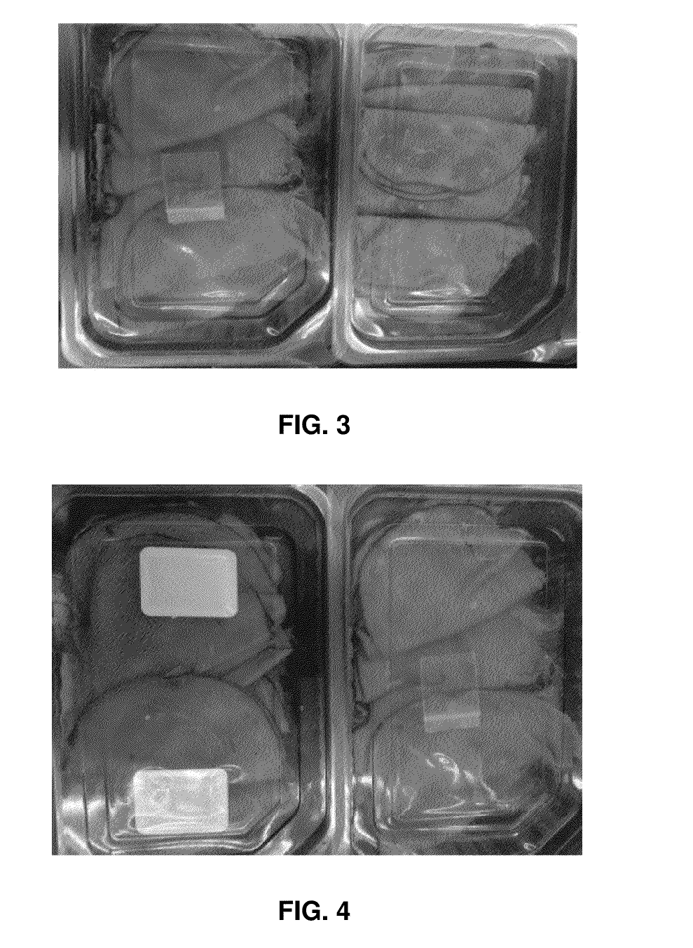

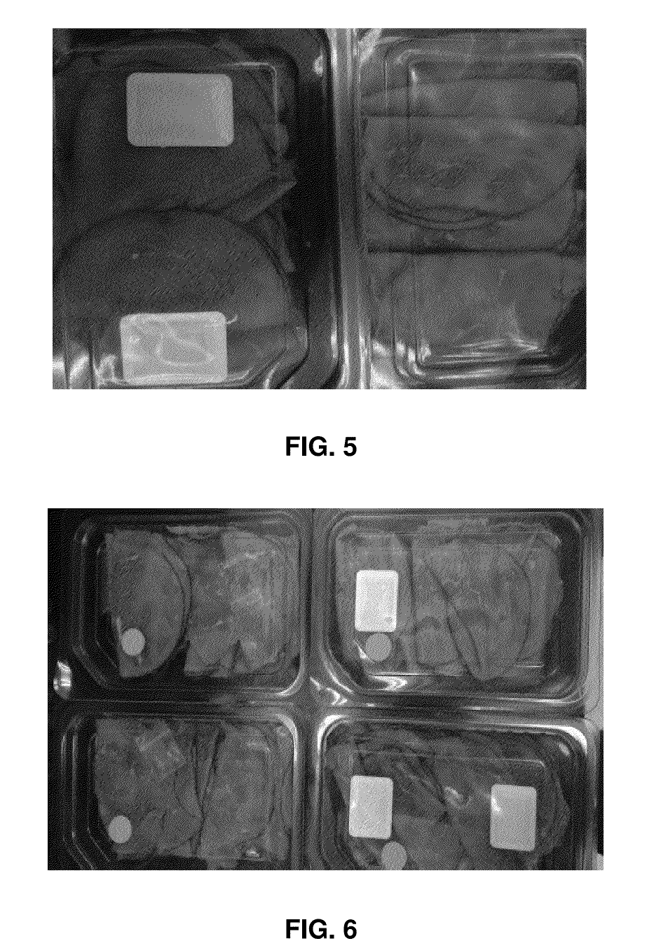

Second roast beef trial. In the second trial, color stability of enzyme samples was intermediate between oxygen scavenger samples (practically no change in a* during light display) and control samples (decrease in redness observed both visually and via a*-values) (FIG. 2). However, there was a rather large variation in a*-values for the enzyme samples. The best looking enzyme sample (E4) is shown in comparison with an average oxygen scavenger sample and an average control sample in FIGS. 3-5 from the end of the storage period.

It is seen from FIGS. 3-5 that both E4 and OS2 have a more even color than C10. In addition, E4 and OS2 had a red color vs. the brownish color of C 10. However, it is also visible that OS2 had a more intense red color than E4. After the end of the actual study, the remaining light stressed samples were put back under the light for an additional six days. Not surprisingly, these additional days caused rapid browning due to the air entering the sample where it was punctured for the O₂-analysis despite being covered by a sticky nickel. Surprisingly, however, some of the enzyme samples held up their color somewhat well. Two of these enzyme samples are shown in FIG. 6 alongside two oxygen scavenger samples, which are shown to have browned completely.

Table 1 below shows the results of the headspace oxygen analysis on day 14. It is seen that samples stored under light had a lower residual oxygen content than samples stored in the dark. The exception was the C SS samples, which also had a higher residual oxygen level, likely due to the fact that one slice of meat has less oxygen consumption than 7 oz. of meat. It is not surprising that the oxygen scavenging stickers did the best job at lowering the residual oxygen level in the packages. The dark control samples (both C DC and E DC) had higher oxygen content than the remaining samples (except C SS). This may be due to the fact that these samples were (nearly) not exposed to light and therefore no photooxidation. The pigment oxidation to myohemichromogen following photooxidation consumes O₂.

TABLE 1
Headspace oxygen content after 14 days of storage
at 4° C. and 150 ft. candles

| Sample | Oxygen content (ppm) | SD |
|---|---|---|
| C | 610 | 28 |
| C SS | 2240 | 1000 |
| E | 465 | 165 |
| OS | 228 | 32 |
| C DC | 2210 | N/A |
| E DC | 2540 | 780 |

Results of the headspace hexanal analysis via solid-phase microextraction gas chromatography are shown in Table 2 below. It is seen that oxidation levels were significantly lower for the control dark always and the enzyme samples than for the single slice control. Due to the addition of nitrite, lipid oxidation levels were, however, rather low in all samples despite the severe discoloration of the single slice control samples.

TABLE 2
Headspace hexanal content of roast beef samples after
14 days of light display (E and C SS) or dark storage (C DA)*

| Sample | Hexanal (ppm) | Std. |
|---|---|---|
| C DA (red) | 0.03 | 0.01 |
| E (intermediate color) | 0.04 | 0.01 |
| C SS (brown) | 0.18 | 0.03 |

*Results are an average of three unique roast beef samples for each treatment.

The results of the NOx analysis are shown in Table 3.

TABLE 3
Residual NaNO₂ and residual NaNO₃ after 14 days of
light display (E and C SS) or dark storage (C DA)*

| Sample | NaNO₂ (ppm) | Std. | NaNO₃ (ppm) | Std. |
|---|---|---|---|---|
| C DA (red) | 4.2 | 0.3 | 22.2 | 1.8 |
| E (intermediate color) | 5.1 | 1.9 | 28.4 | 3.1 |
| C SS (brown) | 3.8 | 0.6 | 16.3 | 1.6 |

*Results are means of duplicate measurements on three individual samples from each treatment, and standard deviations are of the means.

In another experiment, 30 mg glucose, 1 mg glucose oxidase, and 0.1 mg catalase in a 1 ml volume of 50 mM sodium phosphate (pH 6.3) were stored in sealed mason jars flushed with Nitrogen gas to 2-3% 02 in the headspace. A second experiment using 30 mg glucose and 1 mg glucose oxidase in a 1 mL volume of 50 mM, pH 6.3 sodium phosphate buffer, but without catalase, was also stored in the flushed mason jars. As shown in Table 4, glucose oxidase was almost as effects as glucose oxidase when used in combination with catalase.

TABLE 4
Ability of GluOx/CAT and gluOx alone to deplete oxygen

| | O2 depletion (%) | O2 depletion (%) |
|---|---|---|
| | day 5 | day 11 |
| GluOx/CAT | 1.7 | 1.5 |
| GluOx | 1.6 | 1.1 |

### Example 3

### Conclusion

In conclusion, the high level of enzyme (0.1 mg/mL catalase and 1.0 mg/mL glucose oxidase in the sachets) combined with a high level of glucose (3.0%) and the five days in constant darkness prior to light display show promising results concerning improvement of color stability of sliced roast beef.

All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VIII. REFERENCES

The following references, to the extent that they provide exemplary procedural or other details supplementary to those set forth herein, are specifically incorporated herein by reference:
Abe, Y. (1994) Active packaging with oxygen absorbers. In Ahvenainen R, Nattila-Sandholm T, Ohlsson T. Minimal processing of foods. VTT symposium 142, Espoo, pp. 209-233.
Andersen, H. J., Bertelsen, G., Boegh-Soerensen, L., Shek, C. K., & Skibsted, L. H. (1988). Effect of light and packaging conditions on the colour stability of sliced ham. Meat Science, 22, 283-292.
Andersen, H. J., & Rasmussen, M. A. (1992). Interactive packaging as protection against photodegradation of the colour of pasteurized, sliced ham. International Journal of Food Science and Technology, 27, 1-8.
Englander, S. W., Calhoun, D. B., & Englander, J. J. (1987). Biochemistry without oxygen. Analytical Biochemistry, 161, 300-306.
Nakamura, H., Hoshino, J. (1983) Techniques for the preservation of food by employment of an oxygen absorber. Mitsubishi Gas Chemical Co., Tokyo, Ageless® Division, 1-45.
Prabhakar, R., Siegbahn, P. E. M., Minaev, B. F. (2003) Biochimica et Biophysica Acta, 1647, 173-178.
Rooney, M. L. (1995) Active packaging in polymer films. In: Active Food Packaging. London, Blackie Academic Professional.

1. A method of preventing discoloration of a meat product comprising placing a cured meat product in container having a low oxygen environment, wherein said low oxygen environment further comprises a buffered solution comprising at least one oxygen-scavenging enzyme and at least 1% by weight of glucose, and wherein said buffered solution does not come into physical contact with said cured meat product.

2. The method of claim 1, wherein said at least one oxygen-scavenging enzyme is glucose oxidase.

3. The method of claim 2, wherein said buffered solution further comprises catalase.

4. The method of claim 3, wherein the weight ratio of glucose oxidase to catalase is about 10:1.

5. The method of claim 3, wherein the amount of catalase present is about 0.1-0.5 mg/ml, and the amount of glucose oxidase present is about 1.0-5.0 mg/ml.

6. The method of claim 1, wherein said low oxygen environment is a nitrogen gas-flushed environment.

7. The method of claim 1, wherein said container is formed by a rigid or flexible polymer packaging material.

8. The method of claim 7, wherein said buffered solution is located in an air-permeable sachet located in said container, or in the rigid or flexible polymer packaging material or a film applied thereto.

9. (canceled)

10. The method of claim 1, wherein said meat product is a cured meat product.

11. The method of claim 1, wherein said meat product is an uncured meat product.

12-13. (canceled)

14. The method of claim 14, wherein said glucose is present at about 1-3%, about 1-6%, about 3-6%, about 1-10% or about 6-10% by weight of said solution.

15. The method of claim 1, wherein said buffered solution is a phosphate buffered solution and/or said buffered solution is at about pH 6.2-6.4.

16. A container comprising a low oxygen environment, wherein said low oxygen environment further comprises a buffered solution comprising at least one oxygen-scavenging enzyme and at least 1% by weight of glucose.

17. The container of claim 16, wherein said at least one oxygen-scavenging enzyme is glucose oxidase.

18. The container of claim 17, wherein said buffered solution further comprises catalase.

19. The container of claim 18, wherein the weight ratio of glucose oxidase to catalase is about 10:1.

20. The container of claim 18, wherein the amount of catalase present is about 0.1-0.5 mg/ml, and the amount of glucose oxidase present is about 1.0-5.0 mg/ml.

21. The container of claim 16, wherein said low oxygen environment is a nitrogen gas-flushed environment.

22. The container of claim 16, wherein said container is formed by a rigid or flexible polymer packaging material.

23. The container of claim 22, wherein said buffered solution is located in an air-permeable sachet located in said container, or in the rigid or flexible polymer packaging material or a film applied thereto.

24-25. (canceled)

26. The container of claim 16, wherein said glucose is present at about 1-3%, about 1-6%, about 3-6%, about 1-10% or about 6-10% by weight of said solution.

27. The container of claim 16, wherein said buffered solution is a phosphate buffered solution, and/or is at about pH 6.2-6.4.

28. The container of claim 16, further comprising a meat product located in said low oxygen environment, wherein said meat product does not come into physical contact with said buffered solution.

29-30. (canceled)

31. A method of preparing a packaged meat comprising:
(a) providing a container defined by a rigid or flexible polymer packaging material that comprises a buffered solution comprising at least one oxygen-scavenging enzyme and at least 1% by weight of glucose;
(b) reducing the oxygen content inside said container to produce a low oxygen environment;
(c) introducing said meat product into said low oxygen environment such that said buffered solution does not come into physical contact with the cured meat product; and
(d) sealing said container to substantially prevent movement of oxygen into said low oxygen environment.

32-46. (canceled)

47. A buffered solution comprising glucose oxidase and/or catalase.

48-55. (canceled)
